(19) United States
(12) Patent Application Publication
(10) Pub. No.: US 2007/0232824 A1
(43) Pub. Date: Oct. 4, 2007

(54) Heterogeneous catalysis solid base catalyst and a preparation process and a use thereof

(76) Inventors: Yuhan Sun, Beijing (CN); Wei Wei, Beijing (CN); Desheng Zhang, Beijing (CN); Dachuan Shi, Beijing (CN); Liming Liu, Beijing (CN); Hui Wang, Beijing (CN); Huanren Zhang, Beijing (CN); Yanhua Cheng, Beijing (CN); Ning Zhao, Beijing (CN); Mouhua Wang, Beijing (CN); Xiuzhi Wang, Beijing (CN)

Correspondence Address: SOFER & HAROUN LLP., 317 MADISON AVENUE, SUITE 910, NEW YORK, NY, 10017, US

(21) Appl. No.: 11/655,050

(22) Filed: Jan. 18, 2007

(30) Foreign Application Priority Data
Jan. 20, 2006 (FR) CN/200610001363.2

(51) Int. Cl. C07C 69/96; B01J 21/06; B01J 23/02

(52) U.S. Cl. 560/1; 502/340

(57) ABSTRACT

The present invention relates to a solid base catalyst including 10.0%-30.0% by weight of calcium oxide and 70.0%-90.0% by weight of zirconium oxide, in which the calcium source is CaCl₂ or Ca(NO₃)₂, and the Zirconium source is ZrOCl₂ or ZrO(NO₃)₂. The invention also provides a process for the preparation of the catalyst and a use of the catalyst in synthesizing dimethyl carbonate via heterogeneous catalysis. The catalyst shows good catalytic stability and catalytic activity, and can be separated from products easily and is reusable.

### RELATED APPLICATION

This application is related to and claims the benefit of priority from Chinese Patent Application No. CN200610001363.2, filed on Jan. 20, 2006, the entirety of which is incorporated herein by reference.

### FIELD OF THE INVENTION

The invention relates to a heterogeneous catalysis solid base catalyst and a preparation process and a use thereof, in particular to a solid base catalyst for synthesizing dimethyl carbonate by heterogeneous catalysis, and a preparation process and a use thereof.

### BACKGROUND OF THE INVENTION

At present, green chemistry draws wide attention increasingly. Dimethyl Carbonate (abbreviated as DMC), as a kind of new environmental friendly chemical material with low contamination and easy biodegradation, has attracted wide attention on its synthesis and application throughout the world for recent ten years. The industrial processes for synthesizing dimethyl carbonate mainly include phosgenation, methanol oxidation carbonylation, and transesterification. Among them, the transesterification becomes a promising process, because its reaction conditions are moderate, the process procedure is simple, there is no corrosion to equipment and yield is high, also it can produce ethanediol or propane diol at the same time. Of many catalysts used in dimethyl carbonate synthesis by transesterification, solid base catalyst is the main research trend because of its good catalytic effect and easy separation.

The compounded catalyst developed from aldehyde resin and alkaline earth metal carbonate by Wei Tong et.al (Chinese patent application No: 01130469.3) has high reactivity, the catalytic reaction is fast, and requires low reaction temperature. however because of the restriction of reaction equilibrium in the autoclave reactor, the conversion of the ethylene carbonate (EC) after reacting under stirring at 80° C. for 1 hour is 52.9%. When the catalyst is used in a catalytic rectification unit, the activity is high at the beginning of the reaction, under conditions that reaction temperature is 64° C., feed space velocity of EC is 0.3 h⁻¹ and reflux ratio is 6:1, the conversion of EC reaches to 97.4% after reaction for 12 hours. But after reaction for 20 hours, the activity of the catalyst drops, the conversion of EC implies only 63.5% during the reaction. This may be caused by the loss of active components during the reaction.

### SUMMARY OF THE INVENTION

The purpose of the invention is to provide a catalyst that can improve the catalytic stability and catalytic activity in the synthesis of dimethyl carbonate from alkene carbonate, and also can be separated easily, also to provide a preparation process and a use thereof.

On the one hand, the invention provides a solid base catalyst including 10.0%-30.0% by weight of calcium oxide and 70.0%-90.0% by weight of zirconium oxide.

In a preferable embodiment, Ca and Zr as active components in the solid base catalyst of the invention form a solid solution.

On the other hand, the invention also provides a process for the preparation of the solid base catalyst, which comprises adding a mixed solution of calcium source and zirconium source as well as ammonia water solution into a reactor under heating and stirring, and after the completion of the precipitation, making the precipitate undergo aging, washing, drying, and roasting to get the catalyst. Preferably the calcium source is CaCl₂ or Ca(NO₃)₂; and zirconium source is ZrOCl₂ or ZrO(NO₃)₂.

In a preferable embodiment, the process for the preparation of the solid base catalyst of the invention comprises adding a mixed solution of ZrOCl₂ and CaCl₂ or ZrO(NO₃)₂ and Ca(NO₃)₂ as well as ammonia water solution into a reactor under heating and stirring, and after the completion of the precipitation, making the precipitate undergo aging, washing, drying, and roasting to get the catalyst.

In preparation process of the invention, it is preferable that the cation concentration of the mixed solution is in the range of 0.1M-0.5M, and the mole ratio of Zr and Ca is in the range of 1.0-4.1. In another preferable embodiment, the ammonia water solution is the ammonia water solution of 20-50% by volume formulated from distilled water and ammonia water. In still another preferable embodiment, the precipitate forms at 30-70° C., the Ph of the system is controlled in the range of 9-12. It is also preferable in the preparation process of the invention that the aging temperature is in the range of 30-70° C., the time for aging is from 2 to 8 hours, the drying temperature is from 100-110° C., the roasting temperature is from 500 to 800° C., and the time for roasting is from 2 to 6 hours.

In a preferable embodiment of the invention, the preparation process of the invention comprises formulating a calcium source and a zirconium source into a mixed solution having cation concentration of 0.1-0.5M; forming an ammonia water solution of 20-50% by volume using distilled water and ammonia water; under continuous heating and stirring, adding dropwise the mixed solution and the ammonia water simultaneously into a reactor for precipitation, keeping the PH of the precipitate in a range from 9 to 12, and controlling the temperature at 30-70° C., after complete precipitation, aging the precipitate at 30-70° C. for 2-8 hours, the repeatedly washing the precipitate by deionized water until there is no anion, drying the resulted precipitate at 100-110° C. and then roasting it at 500-800° C. for 2-6 hours to obtain the catalyst.

The invention also relates to a use of the solid base catalyst of the invention in reactive rectification process. Preferably the reactive rectification process is applied for the preparation of dimethyl-carbonate using raw materials of alkene carbonate and methanol, more preferably the alkene carbonate is ethylene carbonate or propylene carbonate.

In a further aspect, the invention relates to a reactive rectification process, wherein said reactive rectification process employs the solid base catalyst of the invention, and the reactive rectification process is carried out in a packed tower including rectifying section, reacting section and stripping section, wherein the operational conditions are as follows:
feed space velocity of the alkenecarbonate is in the range of 0.05-0.3 h⁻¹;
reflux ratio at tower top is in the range of 2:1-10:1;
reaction pressure is in the range of 0.2-0.7 MPa;
temperature of reacting section is in the range of 120-160° C.;
Temperature of rectifying section is in the range of 120-140° C.; and
temperature of tower vessel is in the range of 120-140° C.

In a specific embodiment of preparation process of the invention, a process of the invention comprises following steps:

(1) Formulating a mixed solution having cation concentration of 0.1-0.5M from ZrOCl₂ and CaCl₂ or ZrO(NO₃)₂ and Ca(NO₃)₂, in which the mole ratio of ZrOCl₂ and CaCl₂ or ZrO(NO₃)₂ and Ca(NO₃)₂ is from 1.0 to 4.1; formulating an ammonia water solution of 20-50% by volume from distilled water and ammonia water;

(2) Adding dropwise the mixed solution and the ammonia water solution into a reactor for precipitating under continuous heating and stirring, and controlling the pH in the range of 9-12 and temperature in 30-70° C. until the precipitation is completed.

(3) After the completion of the precipitation, aging the precipitate at 30-70° C. for 2-8 hours, and then repeatedly washing it with deionized water until no Cl⁻ is detected, and

(4)Drying the obtained precipitate at 100-110° C. and then roasting it at 500-800° C. for 2-6 hours to obtain the catalyst.

The preparation process employed in the invention to preparing the catalyst is a stepwise precipitation, which completely differs from traditional immersion process and co-precipitation process. On the other hand, conditions for preparing the catalyst are controlled strictly, such that the active components Ca and Zr form a solid solution, interaction between active components becomes more intensive, and new active sites are formed. Defects that active components of the catalyst only form simple complex oxide, there is no interaction between components, and the use of the catalyst is only the sum of uses of two metal oxides are overcome. Not only the activity, selectivity and stability of the catalyst are improved, also the catalyst can better fit the catalytic rectification unit.

Taking alkene carbonate and methanol as raw materials, dimethyl carbonate can be prepared by a reactive rectification process using the solid base catalyst of the invention. Especially the alkene carbonate means ethylene carbonate or propylene carbonate. The detailed procedures are as follows:

The catalyst obtained according to the invention is directly crushed into 2-1.2 mm for use in a reactive rectification process, preferably for use in a pressurized catalytic rectification process.

The reactive rectification process of the invention takes place in a packed tower, applying the catalyst made by the process of the invention. The packed tower includes three sections as rectifying section, reacting section and stripping section; the reactive rectification process mainly takes place in the reacting section of the tower, wherein the operational conditions are as follows:
1) feed space velocity of the alkenecarbonate is in the range of 0.05-0.3 h⁻¹;
2) reflux ratio at tower top is in the range of 2:1-10:1;
3) reaction pressure is in the range of 0.2-0.7 MPa;
4) temperature of reacting section is in the range of 120-160° C.;
5) temperature of rectifying section is in the range of 120-140° C.; and
6) temperature of tower vessel is in the range of 120-140° C.

After reaction for 12-600 hours, products from the tower top and the bottom of the vessel are analyzed by vapor phase chromatography.

Analytic Conditions:

GC-950 gas chromatograph and thermal conductivity cell detector made by Shanghai HaiXin Chromatographic Instruments Co., Ltd. is used for specimen analysis. Chromatographic analytic conditions are as follow : a 2 m long stainless steel tube having internal diameter of 3 mm is used as chromatographic column with supporter of a 60-80 mesh GDX-203 organic supporter; having column temperature of 220° C., vaporization temperature of 220° C., detector cell temperature of 220° C. Carrier gas is 50 ml/min H₂, bridge current is 50 mA, and feed of the specimen is 1 ul.

Catalysts used in conventional catalytic rectification processes for preparing dimethyl carbonate are all homogeneous catalysts. These catalysts are costly, are difficult to separating from products and have low productivity. The novel heterogeneous solid base catalyst of the invention further improves the activity and stability of the catalyst, not only fits for synthesizing dimethyl carbonate by transesterfication, but also fits for catalytic rectification reaction process.

Compared with the prior art, the invention has following advantages:

1) The catalyst has excellent stability, and its rate of deactivation is reduced greatly. This advantage will be obvious when used in catalytic rectification units for continuous reaction;

2) The activity of the catalyst is generated during the interaction between active components and carriers, and the active components form stable chemical bonds with carriers, such that the active components are difficult to loss, and are not easily contaminated by CO₂ and H₂O and the like. The defects that general solid base is easily contaminated by CO₂ and H₂O and the like are overcome.

3) The catalyst overcomes the defect that homogeneous catalyst is difficult to be separated, thus the technological process is simplified. In addition, the catalyst is separable from products easily and is reusable.

### EMBODIMENTS

Following examples are used to demonstrate the invention, but not for the purpose of limitation.

### EXAMPLE 1

0.1M CaCl₂ and ZrOCl₂ mixed solution and 20% (by volume) ammonia water solution were prepared respectively, wherein the mole ratio of ZrOCl₂ and CaCl₂ in 0.1M CaCl₂ and ZrOCl₂ mixed solution is 4.1; under continuous heating and agitating, the mixed solution and ammonia water were simultaneously added dropwise into a beaker, the precipitating temperature was controlled at 30° C. and the pH of the precipitate was 10.

After completion of the precipitation, the precipitate was aged at 60° C. for 2 hours, and washed repeatedly until no Cl⁻ was detected; the washed precipitate was baked at 105° C. for 12 hours, then was placed in a muffle furnace for roasting at 500° C. for 6 hours to obtain the catalyst. Experimental analysis showed that in the catalyst, the amount of CaO is 10% by weight, the amount of ZrO₂ is 90% by weight.

The obtained catalyst was crushed into 1.2-2 mm and used in a catalytic rectification unit. When the pressure was up to 0.2 MPa, the temperature of the reacting section was controlled at 120° C. by an externally heater, the temperature at the bottom of the vessel showed 120° C., the temperature of the rectifying section was 120° C.; the feed space velocity of the ethylene carbonate indicated 0.1 h⁻¹; the reflux ratio was 3:1; the reaction was carried out for 12 hours. Products from top of the tower and from the bottom of the vessel were used for gas phase chromatography analysis. See table 1 for results.

### EXAMPLE 2

0.5M CaCl₂ and ZrOCl₂ mixed solution and 20% (by volume) ammonia water solution were prepared respectively, wherein the mole ratio of ZrOCl₂ and CaCl₂ in 0.5M CaCl₂ and ZrOCl₂ mixed solution is 1.0; under continuous heating and agitating, the mixed solution and ammonia water were simultaneously added dropwise into a beaker, the precipitating temperature was controlled at 30° C. and the pH of the precipitate was 12. After completion of the precipitation, the precipitate was aged at 40° C. for 4 hours, and washed repeatedly until no Cl⁻ was detected; the washed precipitate was baked at 1 00C for 12 hours, then was placed in a muffle furnace for roasting at 600° C. for 4 hours to obtain the catalyst. Experimental analysis showed that in the catalyst, the amount of CaO is 30% by weight, the amount of ZrO₂ is 70% by weight.

The obtained catalyst was crushed into 1.2-2 mm and used in a catalytic rectification unit. When the pressure was 0.3 MPa, the temperature of the reacting section was controlled at 130° C. by an externally heater, the temperature at the bottom of the vessel was 140° C., the temperature of the rectifying section showed 125° C.; the feed space velocity of the ethylene carbonate indicated 0.05 h⁻¹; the reflux ratio was 4:1; the reaction was carried out for 12 hours. Products from top of the tower and from the bottom of the vessel were used for gas phase chromatography analysis. See table 1 for results.

### EXAMPLE 3

0.1M Ca(NO₃)₂ and ZrO(NO₃)₂ mixed solution and 20% (by volume) ammonia water solution were prepared respectively, wherein the mole ratio of ZrO(NO₃)₂ and Ca(NO₃)₂ in 0.1M Ca(NO₃)₂ and ZrO(NO₃)₂ mixed solution was 4.1; under continuous heating and agitating, the mixed solution and ammonia water were simultaneously added dropwise into a beaker, the precipitating temperature was controlled at 60° C. and the pH of the precipitate was 9. After completion of the precipitation, the precipitate was aged at 40° C. for 6 hours, and washed repeatedly until no Cl⁻ was detected; the washed precipitate was baked at 105° C. for 12 hours, then was placed in a muffle furnace for roasting at 700° C. for 4 hours to obtain the catalyst. Experimental analysis showed that in the catalyst, the amount of CaO is 10% by weight, the amount of ZrO₂ is 90% by weight.

The obtained catalyst was crushed into 1.2-2 mm and used in a catalytic rectification unit. When the pressure was 0.6 MPa, the temperature of the reacting section was controlled at 160° C. by an externally heater, the temperature at the bottom of the vessel was 140° C., the temperature of the rectifying section was 135° C.; The feed space velocity of the ethylene carbonate was 0.1 h⁻¹; the reflux ratio was 4:1; the reaction was carried out for 36 hours. Products from the top of the tower and from the bottom of the vessel were used for gas phase chromatography analysis. See table 1 for results.

### EXAMPLE 4

0.15M CaCl₂ and ZrOCl₂ mixed solution and 20% (by volume) ammonia water solution were prepared respectively, wherein the mole ratio of ZrOCl₂ and CaCl₂ in 0.15M CaCl₂ and ZrOC₂ mixed solution is 2.6; under continuous heating and agitating, the mixed solution and ammonia water were simultaneously added dropwise into a beaker, the precipitating temperature was controlled at 40° C. and the pH was 10. After completion of the precipitation, the precipitate was aged at 40° C. for 8 hours, and washed repeatedly until no Cl⁻ was detected; the washed precipitate was baked at 105° C. for 12 hours, then was placed in a muffle furnace for roasting at 800° C. for 2 hours to obtain the catalyst. Experimental analysis showed that in the catalyst, the amount of CaO is 15% by weight, the amount of ZrO₂ is 85% by weight.

The obtained catalyst was crushed into 1.2-2 mm and used in a catalytic rectification unit. When the pressure was 0.7 MPa, the temperature of the reacting section was controlled at 150° C. by an externally heater, the temperature at the bottom of the vessel was 140° C., the temperature of the rectifying section was 140° C.; The feed space velocity of the ethylene carbonate was 0.1 h⁻¹; the reflux ratio was 8:1; the reaction was carried out for 200 hours. Products from top of the tower and from the bottom of the vessel were used for gas phase chromatography analysis. See table 1 for results.

### EXAMPLE 5

0.12M CaCl₂ and ZrOCl₂ mixed solution and 20% (by volume) ammonia water solution were prepared respectively, wherein the mole ratio of ZrOCl₂ and CaCl₂ in 0.12M CaCl₂ and ZrOCl₂ mixed solution is 4.1; under continuous heating and agitating, the mixed solution and ammonia water were simultaneously added dropwise into a beaker, the precipitating temperature was controlled at 40° C. and the pH of the precipitate was 10. After completion of the precipitation, the precipitate was aged at 40° C. for 4 hours, and washed repeatedly until no Cl⁻ was detected; the washed precipitate was baked at 105° C. for 12 hours, then was placed in a muffle furnace for roasting at 800° C. for 4 hours to obtain the catalyst. Experimental analysis showed that in the catalyst, the amount of CaO is 10% by weight, the amount of ZrO₂ is 90% by weight.

The obtained catalyst was crushed into 1.2-2 mm and used in a catalytic rectification unit. When the pressure was 0.5 MPa, the temperature of the reacting section was controlled at 160° C. by an externally heater, the temperature at the bottom of the vessel was 130° C., the temperature of the rectifying section was 130° C.; the feed space velocity of the ethylene carbonate was 0.1 h⁻¹; the reflux ratio was 6:1; the reaction was carried out for 600 hours. Products from top of the tower and from the bottom of the vessel were used for gas phase chromatography analysis. See table 1 for results.

### EXAMPLE 6

0.5M Ca(NO₃)₂ and ZrO(NO₃)₂ mixed solution and 20% (by volume) ammonia water solution were prepared respectively, wherein the mole ratio of ZrO(NO₃)₂ and Ca(NO₃)₂ in 0.5M ZrO (NO3)₂ and Ca(NO₃)₂ mixed solution is 1.0; under continuous heating and agitating, the mixed solution and ammonia water were simultaneously added dropwise into a beaker, the precipitating temperature was controlled at 40° C. and the pH of the precipitate was 10. After completion of the precipitation, the precipitate was aged at 60° C. for 2 hours, and washed repetitively until no Cl⁻ was detected; the washed precipitate was baked at 105° C. for 12 hours, then was placed in a muffle furnace for roasting at 800° C. for 4 hours to obtain the catalyst. Experimental analysis showed that in the catalyst, the amount of CaO is 10% by weight, the amount of ZrO₂ is 90% by weight.

The obtained catalyst was crushed into 1.2-2 mm and used in a catalytic rectification unit. When the pressure was 0.3 MPa, the temperature of the reacting section was controlled at 125° C. by an externally heater, the temperature at the bottom of the vessel was 125° C., the temperature of the rectifying section was 130° C.; the feed space velocity of the ethylene carbonate was 0.3 h⁻¹; the reflux ratio was 10:1; the reaction was carried out for 100 hours. Products from top of the tower and from the bottom of the vessel were used for gas phase chromatography analysis. See table 1 for results.

### EXAMPLE 7

0.15M Ca(NO₃)₂ and ZrO(NO₃)₂ mixed solution and 25% (by volume) ammonia water solution were prepared respectively, wherein the mole ratio of ZrO(NO₃)₂ and Ca(NO₃)₂ in 0.15M Ca(NO₃)₂ and ZrO(NO₃)₂ mixed solution is 2.6; under continuous heating and agitating, the mixed solution and ammonia water were simultaneously added dropwise into a beaker, the precipitating temperature was controlled at 50° C. and the pH of the precipitate was 10. After completion of the precipitation, the precipitate was aged at 50° C. for 3 hours, and washed repeatedly until no Cl⁻ was detected; the washed precipitate was baked at 105° C. for 12 hours, then was placed in a muffle furnace for roasting at 800° C. for 4 hours to obtain the catalyst. Experimental analysis showed that in the catalyst, the amount of CaO is 15% by weight; the amount of ZrO₂ is 85% by weight.

The obtained catalyst was crushed into 1.2-2 mm and used in a catalytic rectification unit. When the pressure was 0.6 MPa, the temperature of the reacting section was controlled at 150° C. by an externally heater, the temperature at the bottom of the vessel was 140° C., the temperature of the rectifying section was 135° C.; The feed space velocity of the ethylene carbonate was 0.2 h⁻¹; the reflux ratio was 4:1; the reaction was carried out for 200 hours. Products from top of the tower and from the bottom of the vessel were used for gas phase chromatography analysis. See table 1 for results.

### EXAMPLE 8

The amount of CaO in the catalyst is 20% by weight. 0.25M CaCl₂ and ZrOCl₂ mixed solution and 35% (by volume) ammonia water solution were prepared respectively, wherein the mole ratio of ZrOCl₂ and CaCl₂ in 0.25M CaCl₂ and ZrOCl₂ mixed solution is 1.8; under continuous heating and agitating, the mixed solution and ammonia water were simultaneously added dropwise into a beaker, the precipitating temperature was controlled at 40° C. and the pH of the precipitate was 11. After completion of the precipitation, the precipitate was aged at 50° C. for 4 hours, and was washed repeatedly until no Cl⁻ was detected; the washed precipitate was baked at 105° C. for 12 hours, then was placed in a muffle furnace for roasting at 800° C. for 4 hours to obtain the catalyst. Experimental analysis showed that in the catalyst, the amount of CaO is 20% by weight; the amount of ZrO₂ is 80% by weight.

The obtained catalyst was crushed into 1.2-2 mm and used in a catalytic rectification unit. When the pressure was 0.5 MPa, the temperature of the reacting section was controlled at 160° C. by an externally heater, the temperature at the bottom of the vessel was 140° C., the temperature of the rectifying section was 140° C.; The feed space velocity of the ethylene carbonate was 0.1 h⁻¹; the reflux ratio was 4:1; the reaction was carried out for 150 hours. Products from top of the tower and from the bottom of the vessel were used for gas phase chromatography analysis. See table 1 for results.

TABLE 1
Results of the reactions

| | | DMC Space-time yield |
|---|---|---|
| | EC Conversion (%) | (g.g⁻¹h⁻¹) |
| Example 1 | 69.7 | 0.009 |
| Example 2 | 77.8 | 0.011 |
| Example 3 | 88.7 | 0.016 |
| Example 4 | 89.3 | 0.017 |
| Example 5 | 92.5 | 0.023 |
| Example 6 | 93.9 | 0.024 |
| Example 7 | 90.9 | 0.018 |
| Example 8 | 93.4 | 0.025 |

### EXAMPLE 9-16

Procedures of examples 1-8 are repeated, except that ethylene carbonate was replaced by propylene carbonate. The obtained results were provided in Table 2.

TABLE 2
Results of the reactions

| | | DMC Space-time yield |
|---|---|---|
| | PC Conversion (%) | (g.g⁻¹h⁻¹) |
| Example 9 | 71.2 | 0.008 |
| Example 10 | 76.9 | 0.010 |
| Example 11 | 86.8 | 0.015 |
| Example 12 | 88.2 | 0.015 |
| Example 13 | 90.1 | 0.022 |
| Example 14 | 92.5 | 0.023 |
| Example 15 | 91.3 | 0.016 |
| Example 16 | 94.7 | 0.026 |

What is claimed is:

1. A solid base catalyst comprising:
10.0%-30.0% by weight of calcium oxide; and
70.0%-90.0% by weight of zirconium oxide.

2. The solid base catalyst according to claim 1, wherein calcium and zirconium as active components form a solid solution.

3. A process for the preparation of the catalyst according to claim 1 comprising:
formulating a calcium source and a zirconium source into a mixed solution having cation concentration of 0.1-0.5M;
forming an ammonia water solution of 20-50% by volume using distilled water and ammonia water;
under continuous heating and stirring, adding dropwise the mixed solution and the ammonia water simultaneously into a reactor for precipitation, keeping the pH of precipitate in a range from 9 to 12, and
controlling the temperature at 30-70° C., after complete precipitation, aging the precipitate at 30-70° C. for 2-8 hours, then repeatedly washing the precipitate by deionized water until there is no anion, drying the resulted precipitate at 100-110° C. and then roasting the product at 500-800° C. for 2-6 hours to obtain the catalyst.

4. The process according to claim 3, wherein the calcium source is CaCl₂ or Ca(NO₃)₂, the zirconium source is ZrOCl₂ or ZrO(NO₃)₂.

5. The process according to claim 3, wherein the cation concentration of the mixed solution is 0.1M -0.5M, and the mole ratio of Zr and Ca is 1.0-4.1.

6. A use of the catalyst according to claim 1 in reactive rectification process.

7. The use of claim 6, wherein the reactive rectification process is adopted for the preparation of dimethyl carbonate using alkene carbonate and methanol as raw materials.

8. The use of claim 7, wherein the alkene carbonate is ethylene carbonate or propylene carbonate.

9. A reactive rectification process, wherein said reactive rectification process employs the catalyst of claim 1, and the reactive rectification process is carried out in a packed tower including rectifying section, reacting section and stripping section, wherein the operational conditions are as follows:
feed space velocity of the alkenecarbonate is in the range of 0.05-0.3 h⁻¹;
reflux ratio at tower top is in the range of 2:1-10:1;
reaction pressure is in the range of 0.2-0.7 MPa;
temperature of reacting section is in the range of 120-160° C.;
temperature of rectifying section is in the range of 120-140° C.; and
temperature of tower vessel is in the range of 120-140° C.

10. The reactive rectification process of claim 9, wherein the catalyst is crushed directly into 2-1.2 mm for use.

11. The reactive rectification process of claim 8, wherein the process is a pressurized catalytic rectification process.
